(19) United States
(12) Patent Application Publication
(10) Pub. No.: US 2017/0020736 A1
(43) Pub. Date: Jan. 26, 2017

(54) DRESSING COMPRISING ELECTRODES

(71) Applicant: Microarray Limited, Sharnbrook, Bedford (GB)

(72) Inventors: Paul Davis, Sharnbrook, Bedford (GB); Stuart Collyer, Bedford (GB)

(73) Assignee: Microarray Limited, Sharnbrook, Bedford (GB)

(21) Appl. No.: 15/300,983

(22) PCT Filed: Mar. 25, 2015
(86) PCT No.: PCT/GB2015/050887

§ 371 Date: Sep. 30, 2016

(30) Foreign Application Priority Data
Apr. 2, 2014 (GB) 1405912.5

(51) Int. Cl. A61F 13/00; A61N 1/04

(52) U.S. Cl. CPC A61F 13/00063; A61N 1/0468; A61F 13/00072

(57) ABSTRACT

A dressing comprising first and second electrodes, an electrical power supply, and further comprising a physiologically or antimicrobially active precursor substance, the dressing being operable, when placed on a skin site to be treated, for a first treatment period, whereby the electrochemical oxidation or reduction of the precursor substance on one of the electrodes to produce a physiologically active oxidised or reduced substance which is capable of diffusing towards the skin site for the treatment thereof is carried out, and subsequently for a first rest period, the electrochemical oxidation or reduction is stopped, wherein subsequent treatment periods followed by rest periods are carried out over time.

### TECHNICAL FIELD

The present invention relates to a dressing comprising first and second electrodes, an electrical power supply, and further comprising a physiologically or antimicrobially active precursor substance, and a method of operating the dressing.

### BACKGROUND AND PRIOR ART

It has been suggested to use electrochemical processes in skin dressings to produce an active species as desired, which migrates to the surface of the skin through the dressing.

WO 2013/140176 discloses a skin dressing which comprises first and second electrodes and can be connected to a power supply to deliver the active agent.

However, it has been found that electrochemical delivery of an active species from a dressing can occur over a short space of time, delivering all of the available active species. Whilst this may be essential in order to build up a sufficient concentration, e.g. to reach a sufficient kill concentration of antimicrobial action, this can result in a short lifetime of the dressing.

Further improvements in this area would therefore be of great use.

### SUMMARY OF INVENTION

In a first aspect the invention relates to a dressing comprising first and second electrodes, an electrical power supply, and further comprising a physiologically or antimicrobially active precursor substance, the dressing being adapted to be operable, when placed on a skin site to be treated, for a first treatment period, whereby the electrochemical oxidation or reduction of the precursor substance on one of the electrodes to produce a physiologically active oxidised or reduced substance which is capable of diffusing towards the skin site for the treatment thereof is carried out, and subsequently for a first rest period, the electrochemical oxidation or reduction is stopped, wherein a subsequent treatment periods followed by rest periods are carried out over time.

In a second aspect, the invention relates to a method of operating a dressing comprising first and second electrodes, an electrical power supply, and further comprising a physiologically or antimicrobially active precursor substance, the method involving operating the dressing for a first treatment period, whereby the electrochemical oxidation or reduction of the precursor substance on one of the electrodes to produce a physiologically active oxidised or reduced substance which is capable of diffusing towards the skin site for the treatment thereof is carried out, and subsequently for a first rest period, the electrochemical oxidation or reduction is stopped, wherein a subsequent treatment periods followed by rest periods are carried out over time.

Thus, the dressing is operable to deliver an initial period of active substance followed by a rest period. This is then repeated, possibly many times, over the lifetime of the dressing.

The time taken for one treatment period followed by a rest period is considered to be a single treatment cycle.

The invention is primarily concerned with low frequency cycles of treatment. In this respect a treatment cycle is preferable at least 1 minute, more preferably at least 10 minutes, more preferably still at least 1 hour, and in a preferred embodiment is at least 6 hours. However in general, treatment cycles will be less than 48 hours, and from 6 to 36 hours is preferred.

It has been observed that the cycling through a treatment phase followed by a subsequent rest phase as multiple advantages over continuous treatment.

It has been found to be possible to build up a sufficient concentration of active substance in a treatment period without necessarily using up all the available active substance. Thus, the treatment phase can be active for a period sufficient to build up such a critical concentration, wherafter a rest period is initiated.

In general rest periods are longer than treatment periods. Thus, in a preferred embodiment the ratio of the duration of the rest periods to the duration of the treatment periods is from 1:1 to 40:1, more preferably from 2:1 to 30:1.

It has been surprisingly observed that diffusion of a chemical species through a hydrogel from a planar surface tends to be directed away from said surface as opposed to in all directions. This has led to the insight that such an arrangement could be employed to ensure separation of cathodic and anodic species formed during an electrochemical process.

In a preferred embodiment the dressing comprises a pair of first and second substantially planar electrodes, each having a first side and a second side, the first sides providing an electrically active surface and the second sides being non-electrically active, the electrodes being arranged to be substantially parallel to each other, each arranged with their second side facing the other electrode and their first sides facing away from the other electrode, the two electrodes being embedded in a hydrogel material, each electrode further comprising means to form an electrical circuit comprising an electrical power supply, the two electrodes becoming the anode and cathode respectively, and the aqueous medium in the hydrogel. Preferably the electrodes are spaced apart facing each other

In this arrangement, in use anodic species and cathodic species will be produced on the outwardly facing surfaces of the electrodes. It has also been observed that such produced species will diffuse away from the surface of the electrodes in a substantially normal direction, i.e. in opposite directions away from the pair of electrodes. It has been surprisingly found that this directional diffusion away from the electrode surface, provides effective separation between the anodic and cathodic products.

The net effect of this arrangement is that physical separation approaching that which would be obtained in the well-known arrangement with a semi-permeable membrane can be achieved, even though no such physical barrier needs to be present.

Thus, preferably the hydrogel comprises an antimicrobially or physiologically active precursor substance, which can be oxidised or reduced by the electrochemical process on the circuit to generate the active substance.

Another advantage of embedding the electrodes in a hydrogel is that they are protected from being fouled by material from a wound or exuding body fluid, which could act to limit the functionality of the electrodes.

In one preferred embodiment the precursor compound is an iodide salt. When the electrodes are electrically connected to each other the negatively charged iodide ions (anions) migrate to the positively charged working electrode. Once there the iodide donates an electron and is oxidised to iodine. Iodine is a well-known physiologically active compound and a potent antimicrobial agent.

In another preferred embodiment the precursor compound is a sulphate (SO₄²) salt. When the electrodes are electrically connected to each other, the negatively charged sulphate ions (anions) migrate to the positively charged working electrodes. Once there they then donate an electron and are oxidised to peroxodisulphate (S₂O₈²). Peroxodisulphate spontaneously decomposes to produce hydrogen peroxide, a highly potent and reactive physiological or antimicrobial agent.

In another preferred embodiment the precursor substance is a chloride (Cl) salt. When the electrodes are electrically connected to each other the negatively charged chloride ions (anions) migrate to the positively charged working electrodes. Once there they then donate an electron and are oxidised to hypochlorous acid.

It has also been noted that, typically an antimicrobially or physiologically active species will be generated on either the anode or the cathode only. Thus, when embedded in a dressing, the electrode which produces the active species is generally arranged to face the body surface. Thus, the present invention provides just as much control and directionality as electrodes in a co-planar arrangement.

Although metallic electrodes would provide an effective electrical circuit with the least resistance, it has been found that this is undesirable because such electrodes can corrode. It has therefore been discovered that non-metallic electrodes, even with their reduced conductivity in comparison to metallic electrodes, are preferred. Moreover, the reduced conductivity has been found to be advantageous in slowing down the electrochemical reaction so that the antimicrobially or physiologically active species can be delivered at an optimised rate, over a longer period of time. Thus the electrodes are preferably non-metallic, e.g. made from carbon, although a wide variety of non-metallic materials are possible.

The non-electrically active sides of the electrodes can be achieved by placing an electrical insulating material on said sides. For example a polymer material can be adhered to one side of the electrode.

In one preferred embodiment the faces of the two electrodes are in contact. In this embodiment, the two electrodes made of conductive material can be separated by an insulating material to provide both the non-electrically active sides which are in contact.

In a particularly preferred arrangement, the electrodes are formed from printed carbon onto opposing sides of an insulating sheet. A preferred insulating material is PET.

The potential difference applied to the electrodes depends on the redox potential of the species being oxidised or reduced. For example, if iodide is being oxidised then the potential must be greater than +0.55V and if sulphate is being oxidised then it should preferably be greater than 2.0V.

In practice the voltage applied will be greater than the minimum value, to ensure reasonable kinetics for the reaction. Thus, voltages of from 1 to 10 volts, are preferably applied, more preferably from 2 to 5 volts are applied.

The skin dressing is typically packaged for optimal performance prior to use, e.g. being sealed in suitable sterile water-impervious packages, e.g. of laminated aluminium foil.

The hydrogel will typically also form the body surface contacting layer.

Preferably the hydrogel is a chemically cross-linked hydrogel.

The hydrogel can control active species flux rates in numerous ways, including by selection of its physical dimensions (especially depth, affecting diffusion path distance), its extent of cross-linking (affecting the rate of solute diffusion) its water content (less water causing a slower diffusion rate), its composition (with immobilised hydrogen bonding groups slowing hydrogen peroxide movement) and/or its surface architecture at the interface with the target site, e.g. wound site, and/or at the interface with the upper component (affecting the contact surface areas and thereby the rate of transfer into or out of the lower component), e.g., it may have a contoured (possibly corrugated) surface.

Typically, skin or a wound is in direct contact with the hydrogel and can (depending on its chemical composition) act to absorb water and other materials exuded from a wound site, enabling the dressing to perform a valuable and useful function by removing such materials from a wound site.

A typical example of an amorphous hydrated hydrogel formulation is: 15% w/w AMPS (sodium salt), 5% w/w glucose, 0.05% w/w potassium iodide, 0.1% zinc lactate, 0.19% polyethylene glycol diacrylate and 0.01% hydroxycyclohexyl phenyl ketone, with the volume made up to 100% with analytical grade DI water. The reagents are thoroughly mixed and dissolved, then polymerised for between 30-60 seconds, using a UV-A lamp delivering approximately 100 mW/cm², to form the required hydrogel. This may be in the form of a flat sheet or, more conveniently, housed in plastic syringes. The amorphous gel may then be dispensed from a syringe into a target site.

A hydrated hydrogel means one or more water-based or aqueous gels, in hydrated form. A hydrated hydrogel can act to absorb water and other materials exuded from a wound site, enabling the dressing to perform a valuable and useful function by removing such materials from a wound site. The presence of glucose further enhances the osmotic strength of the gel, helping it to take up fluid from the wound, as well as providing an energy source for the cells engaged in healing the wound. The hydrated hydrogel also provides a source of moisture, that can act in use to maintain a wound site moist, aiding healing. The hydrated hydrogel also acts as a source of water, causing release of hydrogen peroxide. Use of a hydrated hydrogel has other benefits as discussed in WO 03/090800.

Suitable hydrated hydrogels are disclosed in WO 03/090800. The hydrated hydrogel conveniently comprises hydrophilic polymer material. Suitable hydrophilic polymer materials include polyacrylates and methacrylates, e.g. available commercially in the form of proprietory sheet hydrogel dressings, including poly 2-acrylamido-2-methylpropane sulphonic acid (polyAMPS) or salts thereof (e.g. as described in WO 01/96422), polysaccharides e.g. polysaccharide gums particularly xanthan gum (e.g. available under the Trade Mark Keltrol), various sugars, polycarboxylic acids (e.g. available under the Trade Mark Gantrez AN-169 BF from ISP Europe), poly(methyl vinyl ether co-maleic anhydride) (e.g. available under the Trade Mark Gantrez AN 139, having a molecular weight in the range 20,000 to 40,000), polyvinyl pyrrolidone (e.g. in the form of commercially available grades known as PVP K-30 and PVP K-90), polyethylene oxide (e.g. available under the Trade Mark Polyox WSR-301), polyvinyl alcohol (e.g. available under the Trade Mark Elvanol), cross-linked polyacrylic polymer (e.g. available under the Trade Mark Carbopol EZ-1), celluloses and modified celluloses including hydroxypropyl cellulose (e.g. available under the Trade Mark Klucel EEF), sodium carboxymethyl cellulose (e.g. available under the Trade Mark Cellulose Gum 7LF) and hydroxyethyl cellulose (e.g. available under the Trade Mark Natrosol 250 LR).

Mixtures of hydrophilic polymer materials may be used in a gel.

In a hydrated hydrogel of hydrophilic polymer material, the hydrophilic polymer material is desirably present at a concentration of at least 1%, preferably at least 2%, more preferably at least 5%, yet more preferably at least 10%, or at least 20%, desirably at least 25% and even more desirably at least 30% by weight based on the total weight of the gel. Even higher amounts, up to about 40% by weight based on the total weight of the gel, may be used.

A preferred hydrated hydrogel comprises poly 2-acrylamido-2-methylpropane sulphonic acid (poly AMPS) or salts thereof, preferably in an amount of about 30% by weight of the total weight of the gel.

The skin-contacting layer can be manufactured by known means. Preferably it is manufactured by the polymerisation of AMPS monomer dissolved at the rate of about 40% w/v in a solution buffered to a pH of about 5.5, containing any further ingredients required for controlling the rate of transmission or reaction of oxidised or reduced chemical substance such as iodine. Typically, the iodide concentration should be about 0.01-0.2% w/v. If a stronger antimicrobial effect is required then the level of iodide should be from about 0.05% to about 0.2% w/v together with a higher applied voltage (e.g. 5.0 volts). Methods for the manufacture of this material are as described in patent number EP1631328.

In addition, the dressing may incorporate one or more other active ingredients such as zinc ions, as disclosed in WO 2004/108917. Zinc ions are known to be an essential nutritional trace element with numerous functions in the growth and repair of living tissues.

Lactate ions may be included in the skin dressing. Lactate ions have a mild buffering effect within the delivery system. Lactate ions are also believed to have an important role in stimulating angiogenesis—the growth and regeneration of new blood vessels.

A source of glucose is preferably included in the skin dressing. In addition to its role as a respiratory substrate, glucose is believed to participate (as a metabolic precursor) in building polysaccharides of various types that form extracellular matrix (ECM), essential to tissue repair and healing. Preferred skin-contacting layers of this sort are disclosed in our European Patent Application No. 04250508.1 and British Patent Application No. 0427444.5.

The invention will now be illustrated, by way of example, and with reference to the following figures, in which:

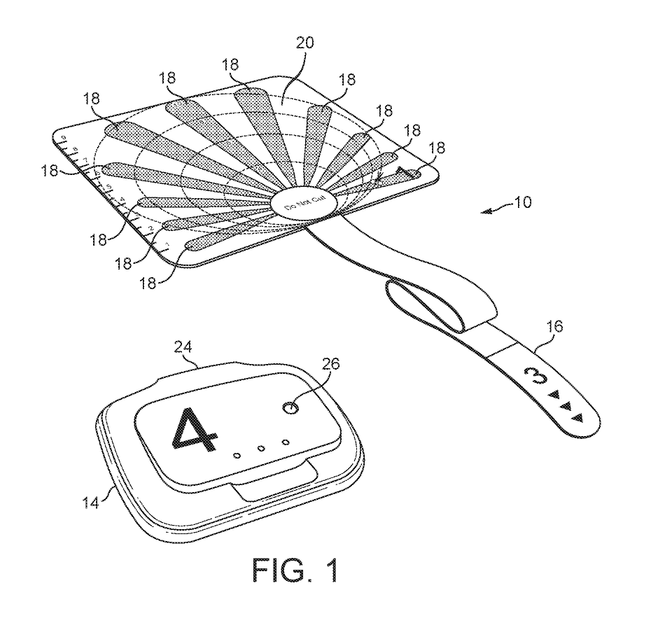

FIG. 1 is an electrochemical dressing for use with the present invention.

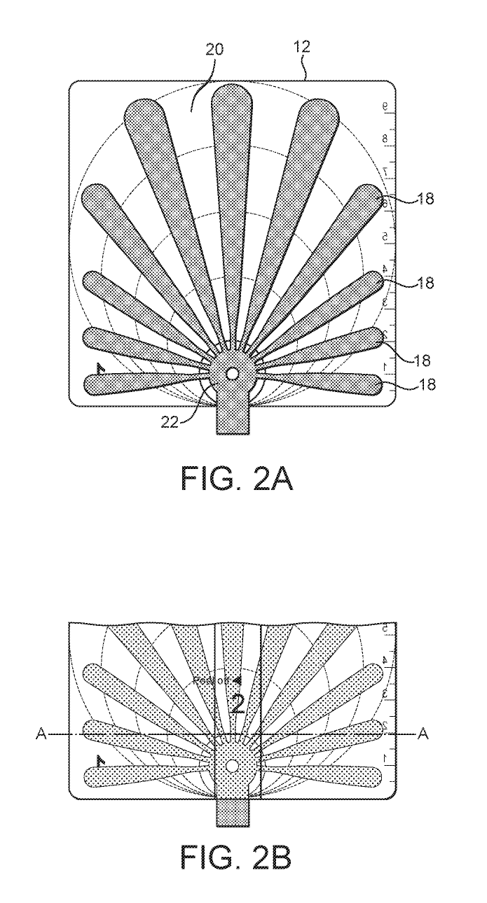

FIGS. 2A and 2B are plan views showing detail of the electrodes of the treatment portion of the dressing shown in FIG. 1.

FIGS. 2C and 2D show side sectional views and exploded views respectively, of the line A-A through FIG. 6B.

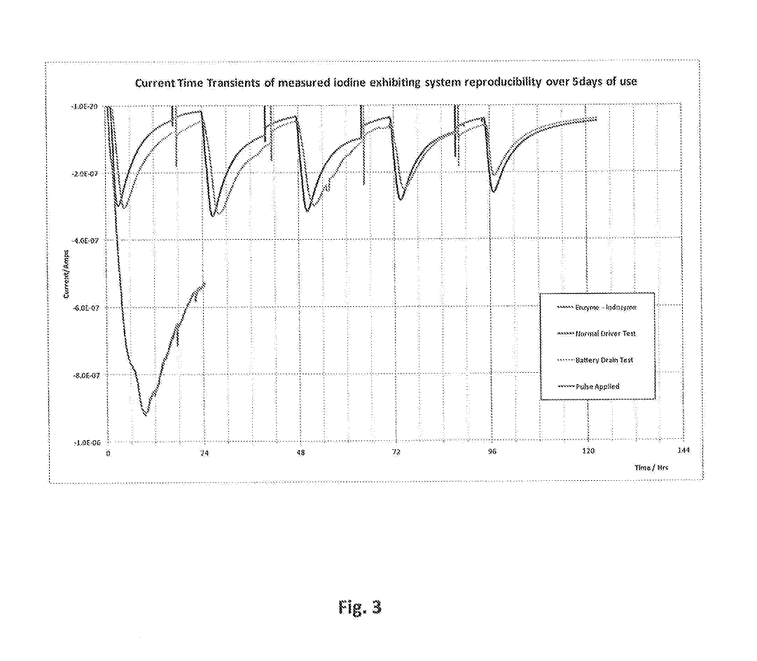

FIG. 3 is a chart of measured current over time illustrating iodine levels.

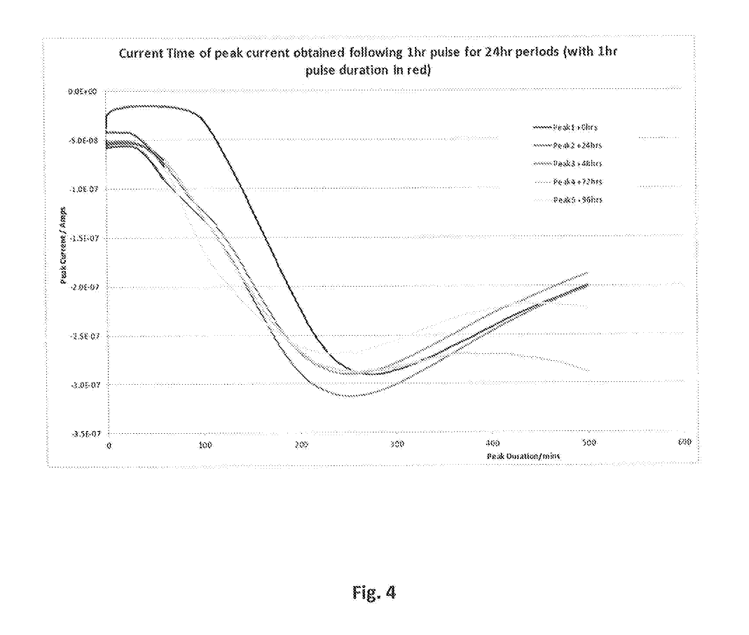

FIG. 4 is a chart of measured current over time illustrating iodine levels.

Turning to the figures, FIG. 1 shows a dressing 10 according to the invention, the dressing being made up of a body treatment portion 12, a remote electrical power portion 14 and an electrical connecting portion 16.

The treatment portion comprises a number of planar electrodes 18 embedded in a cross-linked hydrogel 20. The electrodes meet a central electrode node 22. The body treatment portion 12 is attached to the electrical connecting portion 16 via the electrode node 22.

Electrical power portion has a receiving portion 24 for receiving the electrical connecting portion 16.

When it is desired to apply the dressing 10, the treatment portion is placed on the body surface requiring treatment. The electrical power portion 14 is connected to the electrical receiving portion by feeding it into receiving portion 24. An indicator light 26 lights up when an electrical circuit is formed with the treatment portion 12. The power portion is located, typically on the body, but remote from the treatment area.

Thus, the size and weight of the power portion 14 do not interfere with the functioning of the treatment portion 12.

FIGS. 2A and 2B shows again the detail of the electrode 18 placement in the treatment portion 12, and how they converge at the central electrode node 22.

FIGS. 2C and 2D show in detail how the treatment portion 12 is formed. Shown are releasable backing papers 40 and two hydrogel slabs 42. The electrodes 18 are made up of a PET film 44 onto which has been printed carbon electrically active surfaces 46, 48 forming the cathode and anode respectively.

It can be seen that the cathode and the anode have an electrically active side facing away from the other electrode and an electrically insulating side (the PET side) facing towards the other electrode to form the arrangement of the present invention.

In use, an electrical circuit is made when the power portion 14 is connected to the electrical connecting portion 16. The active precursor substance, e.g. chloride ions, are oxidised at the anode surface 48 to form hypochlorous acid HOCl. The HOCl migrates essentially normal to the surface 48 and therefore directly towards the body surface site 32. Surprisingly the HOCl does not mix with the cathodic reaction products formed on surface 46.

### EXAMPLE

FIG. 3 is a trace of the iodine generation profiles detected in iodine-containing hydrogels subjected to the oxidising action of either the enzyme glucose oxidase or an electrode of the present invention. Deflection of the trace towards the bottom of the graph indicates an increase in iodine generation. The green line shows the over-production of iodine caused by glucose oxidase. The blue lines show the production of iodine by the electrodes at daily intervals over 5 days. The red sections of each electrode trace indicate the time during which the electrical pulse was applied.

FIG. 4 shows the iodine production pulses of FIG. 3, but superimposed on each other in order to make it easier to observe the consistency of iodine production on each of 5 consecutive daily pulses.

It can be seen how the pulsing achieves the necessary kill concentrations over several pulses which extends the lifetime of the dressing to several days, by conserving the supply of iodide over several pulses.

1-5. (canceled)

6. A dressing, comprising:
a body treatment portion comprising first and second electrodes and a physiologically or antimicrobially active precursor substance; and
an electrical power supply; and
wherein when placed on a skin site to be treated the dressing is operable:
for a treatment period, to carry out electrochemical oxidation or reduction of the precursor substance on one of the electrodes to produce a physiologically active oxidised or reduced substance which is capable of diffusing towards the skin site for the treatment thereof;
subsequently for a rest period, to stop the electrochemical oxidation or reduction; and
to carry out subsequent treatment periods followed by subsequent rest periods over time; and
wherein the treatment period and the rest period define a treatment cycle.

7. The dressing of claim 6, wherein the treatment cycle is at least 1 minute.

8. The dressing of claim 6, wherein the treatment cycle is at least 6 hours.

9. The dressing of claim 6, wherein the treatment cycle is from 6 to 36 hours.

10. The dressing of claim 9, wherein a ratio of a duration of the rest period to a duration of the treatment period is from 2:1 to 30:1.

11. The dressing of claim 6, wherein a ratio of a duration of the rest period to a duration of the treatment period is from 1:1 to 40:1.

12. A method of operating a dressing, wherein the dressing comprises an electrical power supply and a body treatment portion comprising first and second electrodes and a physiologically or antimicrobially active precursor substance, the method comprising:
producing, for a treatment period, a physiologically active oxidised or reduced substance which is capable of diffusing towards a skin site for the treatment thereof by carrying out electrochemical oxidation or reduction of the precursor substance on one of the electrodes; and
stopping, for a rest period, the electrochemical oxidation or reduction; and
repeating the producing and the stopping over time;
wherein the treatment period and the rest period define a treatment cycle.

13. The method of claim 12, wherein the treatment cycle is at least 1 minute.

14. The method of claim 12, wherein the treatment cycle is at least 6 hours.

15. The method of claim 12, wherein the treatment cycle is from 6 to 36 hours.

16. The method of claim 15, wherein a ratio of a duration of the rest period to a duration of the treatment period is from 2:1 to 30:1.

17. The method of claim 12, wherein a ratio of a duration of the rest period to a duration of the treatment period is from 1:1 to 40:1.
